(19) United States
(12) Patent Application Publication
(10) Pub. No.: US 2018/0289745 A1
(43) Pub. Date: Oct. 11, 2018

(54) Compositions and Methods for Treating Parkinson's Disease (PD) and Related Disorders

(71) Applicant: Crestovo LLC, Cambridge, MA (US)

(72) Inventor: Thomas Julius BORODY, Five Dock (AU)

(73) Assignee: Crestovo LLC, Cambridge, MA (US)

(21) Appl. No.: 15/606,935

(22) Filed: May 26, 2017

Related U.S. Application Data
(60) Provisional application No. 62/482,162, filed on Apr. 5, 2017.

(51) Int. Cl. A61K 35/37; A61K 9/48; A61K 35/741

(52) U.S. Cl. CPC A61K 35/37; A61K 35/12; A61K 35/741; A61K 9/4875

(57) ABSTRACT

The present disclosure is in the field of pharmaceutical compositions suitable for the treatment of diseases in mammals. The disclosure provides novel compositions comprising non-pathogenic fecal microbes or a sterile fecal filtrate for treating Parkinson's disease and related diseases. The disclosure also provides methods for treating a subject with the compositions disclosed herein.

### CROSS REFERENCE TO RELATED APPLICATIONS

This application claims the benefit of U.S. Provisional Application No. 62/482,162 filed Apr. 5, 2017, the contents of which are herein incorporated by reference in their entirety.

### FIELD

The present disclosure relates to pharmaceutical compositions and methods suitable for treating Parkinson's disease (PD) and related disorders.

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and result in increased susceptibility to pathogens, altered metabolic profiles, or induction of proinflammatory signals that can lead to local or systemic inflammation or autoimmunity. Additionally, such a disrupted microbiota may be infected by incoming pathogen or pathogens, which can cause pain, diarrhea, gas, constipation among other symptoms. Hence. the intestinal microbiota plays a significant role in the pathogenesis of many disorders such as pathogenic infections of the gut.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal tube of human microbiota either in the form of homogenised stool, or cultured stool components such as Clostridia, to implant in the colon and thereby displace or eradicate pathogenic bacteria, e.g., C. difficile.

Parkinson's disease (PD) is a chronic progressive disorder of the nervous system that affects movement. PD develops as neurons in the substantia nigra region of the brain are lost, resulting in reduced availability of the neurotransmitter dopamine. Dopamine levels may also be decreased in the intestines. Nearly one million people in the US are living with PD. PD usually develops around age 60 and risks increase with age. Men are more likely to develop PD than woman. A family history of PD and ongoing exposure to herbicides and pesticides may put a person at increased risk.

PD causes stiffness or slow movements. PD can develop slowly and often times start with a slight tremor of the hand or development of soft or slurred speech. Patients also suffer from postural instability. Gastrointestinal symptoms include feelings of nausea or bloating, difficulty eliminating and constipation.

The severity of PD can be classified as mild, moderate, or advanced. In the mild stage of PD patients present with tremor, changes in facial expression, and possible slight movement symptoms. In the moderate stage patients may show movement symptoms on both sides of the body, including slowed movement, trouble balancing, and episodes when the feet feel “stuck” to the ground. In the advanced stage of PD walking becomes difficult, and hallucinations and delusions are prominent.

There currently exists no cure for PD, however, there are treatment options to manage symptoms. Some medications used to treat PD increase or substitute dopamine in a person's brain. Surgery can be used to regulate regions of the brain and improve symptoms. Although current therapies can alleviate some of the symptoms of PD, they have variable effectiveness, and are accompanied by unwanted side effects. Additionally, the patient's response to these therapies often decreases over time. Thus, there is an unmet medical need for additional drugs or therapies for more effective treatments for PD and related disorders that are easier to administer.

### SUMMARY

The present disclosure provides compositions, methods, and dosing regimens for treating or preventing PD.

In one aspect, the present disclosure provides a method for treating PD in a subject in need thereof, where the method comprises administering to the subject a pharmaceutically active dose of a therapeutic composition comprising or derived from live non-pathogenic fecal bacteria or a sterile fecal filtrate. In one aspect, a sterile fecal filtrate originates from a donor stool. In another aspect, a sterile fecal filtrate originates from cultured microorganisms.

In another aspect, this disclosure provides use of a composition comprising live non-pathogenic fecal bacteria in the manufacture of a medication for the treatment of PD.

In one aspect, the present disclosure provides a method for treating PD in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily or at least once or twice weekly for at least three, eight, ten, or twenty consecutive weeks.

In one aspect, the present disclosure provides a method for treating PD in a subject in need thereof, where the method comprises administering orally to the subject a pharmaceutically active dose of a therapeutic composition comprising a liquid, frozen, lyophilized, or encapsulated sterile fecal filtrate, where the dose is administered at a dosing schedule of at least once or twice daily or at least once or twice weekly for at least three, eight, ten, or twenty consecutive weeks.

In one aspect, a method achieves a remission, cure, response, or resolution rate of PD of at least about 80%. In one aspect, the present disclosure provides a method which eliminates or reduces one or more PD symptoms selected from the group consisting of tremor, slowed movement (bradykinesia), rigid muscles, impaired posture and balance, loss of automatic movements, speech changes, and writing changes.

In an aspect, a fecal microbiota in a therapeutic composition comprises a donor's substantially entire and non-selected fecal microbiota, reconstituted fecal material, synthetic fecal material.

### DETAILED DESCRIPTION

Unless defined otherwise herein, terms are to be understood according to conventional usage by those of ordinary skill in the relevant art.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, “microbiota,” and “flora” refer to a community of microbes that live in or on a subject's body, both sustainably and transiently, including eukaryotes, archaea, bacteria, and viruses (including bacterial viruses (i.e., phage)). A non-selected fecal microbiota refers to a community or mixture of fecal microbes derived from a donor's fecal sample without selection and substantially resembling microbial constituents and population structure found in such fecal sample.

As used herein, a “sterile fecal filtrate” or a “non-cellular fecal filtrate” refers to a liquid component of a fecal material, where the liquid component is free or substantially free of cell-based living organisms (e.g., bacteria, fungi, or their spores), but retains bacteriophages and non-cellular biological materials. Preferably, a non-cellular or sterile fecal filtrate is also free of viruses for eukaryotic host cells.

As used herein, “remission, cure, or resolution rate” refers to the percentage of patients that are cured or obtain remission or complete resolution of a condition in response to a given treatment. Remission, cure, or resolution of PD refers to complete cessation of one or more symptoms that a patient manifests prior to receiving a treatment described here. Such symptoms can include, but are not limited to, tremor, slowed movement (bradykinesia), rigid muscles, impaired posture and balance, loss of automatic movements, speech changes, and writing changes. Each of the foregoing symptoms are further explained below.
Tremor. A tremor, or shaking, usually begins in a limb, often a patient's hand or fingers. A patient may experience a back-and-forth rubbing of their thumb and forefinger, known as a pill-rolling tremor. One characteristic of Parkinson's disease is a tremor of a patient's hand when it is relaxed (at rest).
Slowed movement (bradykinesia). Over time, Parkinson's disease may reduce a patient's ability to move and slow a patient's movement, making simple tasks difficult and time-consuming. A patient's steps may become shorter when they walk, or they may find it difficult to get out of a chair. Also, a patient may drag their feet as them try to walk, making it difficult to move.
Rigid muscles. Muscle stiffness may occur in any part of a patient's body. The stiff muscles can limit a patient's range of motion and cause pain.
Impaired posture and balance. A patient's posture may become stooped, or the patient may have balance problems as a result of Parkinson's disease.
Loss of automatic movements. In Parkinson's disease, a patient may have a decreased ability to perform unconscious movements, including blinking, smiling or swinging their arms when they walk.
Speech changes. A patient may have speech problems as a result of Parkinson's disease. They may speak softly, quickly, slur or hesitate before talking. Their speech may be more of a monotone rather than with the usual inflections.
Writing changes. It may become hard to write, and a patient's writing may appear small.

As used herein, “response rate” refers to the percentage of patients that respond positively (e.g., reduced severity or frequency of one or more symptoms) to a given treatment.

As used herein, the staging of a PD patient is according to the Hoehn and Yahr Staging summarized in Table 1 (see also Hoehn M M, Yahr M D, Parkinsonism: onset, progression and mortality. Neurology 1967, 17:427-42)).

TABLE 1
Hoehn and Yahr Staging of Parkinson's Disease.

I. Stage One: (mild or early disease): Symptoms affect only one side of
the body.
1. Signs and symptoms on one side only
2. Symptoms mild
3. Symptoms inconvenient but not disabling
4. Usually presents with tremor of one limb
5. Friends have noticed changes in posture, locomotion and facial
expression
II. Stage Two: Both sides of the body are affected, but posture remains
normal.
1. Symptoms are bilateral
2. Minimal disability
3. Posture and gait affected
III. Stage Three: (moderate disease): Both sides of the body are affected,
and there is mild imbalance during standing or walking. However, the
person remains independent.
1. Significant slowing of body movements
2. Early impairment of equilibrium on walking or standing
3. Generalized dysfunction that is moderately severe
IV. Stage Four: (advanced disease): Both sides of the body are affected,
and there is disabling instability while standing or walking. The
person in this stage requires substantial help.
1. Severe symptoms
2. Can still walk to a limited extent
3. Rigidity and bradykinesia
4. No longer able to live alone
5. Tremor may be less than earlier stages
V. Stage Five: (Severe) fully developed disease is present. The person is
restricted to a bed or chair.
1. Cachectic stage
2. Invalidism complete
3. Cannot stand or walk
4. Requires constant nursing care

As used herein, “Unified Parkinson Disease Rating Scale” or “UPDRS” refers to a rating tool to follow the longitudinal course of PD and represents the level or severity of PD symptoms (Table 2). UPDRS consists of a three-part test. It is made up of the 1) Mentation, Behavior, and Mood, 2) Activities of Daily Living (ADL) and 3) Motor sections. A total of 31 items are included in Parts I, II and III test. Each item receives a score ranging from 0 to 4 where 0 represents the absence of impairment and 4 represents the highest degree of impairment. The sum of Parts I, II and III at each study visit provides a Total UPDRS score. A total of 199 points are possible. 199 represents the worst (total) disability), 0—no disability. UPDRS is used for measuring the change from baseline in efficacy variables during a treatment described here.

TABLE 2
Unified Parkinson Disease Rating Scale (UPDRS) for
characterizing severity of Parkinson's Disease.

I. Mentation, Behavior, Mood
Intellectual Impairment
0-none
1-mild (consistent forgetfulness with partial recollection of events with
no other difficulties)
2-moderate memory loss with disorientation and moderate difficulty
handling complex problems
3-severe memory loss with disorientation to time and often place, severe
impairment with problems
4-severe memory loss with orientation only to person, unable to make
judgments or solve problems
Thought Disorder
0-none
1-vivid dreaming
2-“benign” hallucination with insight retained
3-occasional to frequent hallucination or delusions without insight, could
interfere with daily activities
4-persistent hallucination, delusions, or florid psychosis.
Depression
0-not present
1-periods of sadness or guilt greater than normal, never sustained for more
than a few days or a week
2-sustained depression for >1 week
3-vegetative symptoms (insomnia, anorexia, abulia, weight loss)
4-vegetative symptoms with suicidality
Motivation/Initiative
0-normal
1-less of assertive, more passive
2-loss of initiative or disinterest in elective activities
3-loss of initiative or disinterest in day to say (routine) activities
4-withdrawn, complete loss of motivation
II. Activities of Daily Living
Speech
0-normal
1-mildly affected, no difficulty being understood
2-moderately affected, may be asked to repeat
3-severely affected, frequently asked to repeat
4-unintelligible most of time
Salivation
0-normal
1-slight but noticeable increase, may have nighttime drooling
2-moderately excessive saliva, hay minimal drooling
3-marked drooling
Swallowing
0-normal
1-rare choking
2-occasional choking
3-requires soft food
4-requires NG tube or G-tube
Handwriting
0-normal
1-slightly small or slow
2-all words small but legible
3-severely affected, not all words legible
4-majority illegible
Cutting Food/Handing Utensils
0-normal
1-somewhat slow and clumsy but no help needed
2-can cut most foods, some help needed
3-food must be cut, but can feed self
4-needs to be fed
Dressing
0-normal
1-somewhat slow, no help needed
2-occasional help with buttons or arms in sleeves
3-considerable help required but can do something alone
4-helpless
Hygiene
0-normal
1-somewhat slow but no help needed
2-needs help with shower or bath or very slow in hygienic care
3-requires assistance for washing, brushing teeth, going to bathroom
4-helpless
Turning in Bed/Adjusting Bed Clothes
0-normal
1-somewhat slow no help needed
2-can turn alone or adjust sheets but with great difficulty
3-san initiate but not turn or adjust alone
4-helpless
Falling-Unrelated to Freezing
0-none
1-rare falls
2-occasional, less than one per day
3-average of once per day
4- >1 per day
Freezing When Walking
0-normal
1-rare, may have start hesitation
2-occasional falls from freezing,
3-frequent freezing, occasional falls
4-frequent falls from freezin
Walking
0-normal
1-mild difficulty, day drag legs or decrease arm swing
2-moderate difficultly requires no assist
3-severe disturbance requires assistance
4-cannot walk at all even with assist
Tremor
0-absent
1-slight and infrequent, not bothersome to patient
2-moderate, bothersome to patient
3-severe, interfere with many activities
4-marked, interferes with many activities
Sensory Complaints Related to Parkinsonism
0-none
1-occasionally has numbness, tingling, and mild aching
2-frequent, but not distressing
3-frequent painful sensation
4-excruciating pain
III. Motor Exam
Speech
0-normal
1-slight loss of expression, diction,volume
2-monotone, slurred but understandable, mod. impaired
3-marked impairment, difficult to understand
4-unintelligible
Facial Expression
0-Normal
1-slight hypomymia, could be poker face
2-slight but definite abnormal diminution in expression
3-mod. hypomimia, lips parted some of time
4-masked or fixed face, lips parted ¼ of inch or more with complete loss
of expression
Tremor at Rest
Face
0-absent
1-slight and infrequent
2-mild and present most of time
3-moderate and present most of time
4-marked and present most of time
Right Upper Extremity (RUE)
0-absent
1-slight and infrequent
2-mild and present most of time
3-moderate and present most of time
4-marked and present most of time
LUE
0-absent
1-slight and infrequent
2-mild and present most of time
3-moderate and present most of time
4-marked and present most of time
RLE
0-absent
1-slight and infrequent
2-mild and present most of time
3-moderate and present most of time
4-marked and present most of time
LLE
0-absent
1-slight and infrequent
2-mild and present most of time
3-moderate and present most of time
4-marked and present most of time
Action or Postural Tremor
RUE
0-absent
1-slight, present with action
2-moderate, present with action
3-moderate present with action and posture holding
4-marked, interferes with feeding
LUE
0-absent
1-slight, present with action
2-moderate, present with action
3-moderate present with action and posture holding
4-marked, interferes with feeding
Rigidity
Neck
0-absent
1-slight or only with activation
2-mild/moderate
3-marked, full range of motion
4-severe
RUE
0-absent
1-slight or only with activation
2-mild/moderate
3-marked, full range of motion
4-severe
LUE
0-absent
1-slight or only with activation
2-mild/moderate
3-marked, full range of motion
4-severe
RLE
0-absent
1-slight or only with activation
2-mild/moderate
3-marked, full range of motion
4-severe
LLE
0-absent
1-slight or only with activation
2-mild/moderate
3-marked, full range of motion
4-severe
Finger taps
Right
0-normal
1-mild slowing, and/or reduction in amp.
2-moderate impaired. Definite and early fatiguing, may have occasional
arrests
3-severely impaired. Frequent hesitations and arrests.
4-can barely perform
Left
0-normal
1-mild slowing, and/or reduction in amp.
2-moderate impaired. Definite and early fatiguing, may have occasional
arrests
3-severely impaired. Frequent hesitations and arrests.
4-can barely perform
Hand Movements (open and close hands in rapid succession)
Right
0-normal
1-mild slowing, and/or reduction in amp.
2-moderate impaired. Definite and early fatiguing, may have occasional
arrests
3-severely impaired. Frequent hesitations and arrests.
4-can barely perform
Left
0-normal
1-mild slowing, and/or reduction in amp.
2-moderate impaired. Definite and early fatiguing, may have occasional
arrests
3-severely impaired. Frequent hesitations and arrests.
4-can barely perform
Rapid Alternating Movements (pronate and supinate hands)
Right
0-normal
1-mild slowing, and/or reduction in amp.
2-moderate impaired. Definite and early fatiguing, may have occasional
arrests
3-severely impaired. Frequent hesitations and arrests.
4-can barely perform
Left
0-normal
1-mild slowing, and/or reduction in amp.
2-moderate impaired. Definite and early fatiguing, may have occasional
arrests
3-severely impaired. Frequent hesitations and arrests.
4-can barely perform
Leg Agility (tap heel on ground, amp should be 3 inches)
Right
0-normal
1-mild slowing, and/or reduction in amp.
2-moderate impaired. Definite and early fatiguing, may have occasional
arrests
3-severely impaired. Frequent hesitations and arrests.
4-can barely perform
Left
0-normal
1-mild slowing, and/or reduction in amp.
2-moderate impaired. Definite and early fatiguing, may have occasional
arrests
3-severely impaired. Frequent hesitations and arrests.
4-can barely perform
Arising From Chair (pt. arises with arms folded across chest)
0-normal
1-slow, may need more than one attempt
2-pushes self up from arms or seat
3-tends to fall back, may need multiple tries but can arise without
assistance
4-unable to arise without help
Posture
0-normal erect
1-slightly stooped, could be normal for older person
2-definitely abnormal, mod. stooped, may lean to one side
3-severely stooped with kyphosis
4-marked flexion with extreme abnormality of posture
Gait
0-normal
1-walks slowly, may shuffle with short steps, no festination or propulsion
2-walks with difficulty, little or no assistance, some festination, short steps
or propulsion
3-severe disturbance, frequent assistance
4-cannot walk
Postural Stability (retropulsion test)
0-normal
1-recovers unaided
2-would fall if not caught
3-falls spontaneously
4-unable to stand
Body Bradykinesia/Hypokinesia
0-none
1-minimal slowness, could be normal, deliberate character
2-mild slowness and poverty of movement, definitely abnormal, or dec.
amp. of movement
3-moderate slowness, poverty, or small amplitude
4-marked slowness, poverty, or amplitude

As used herein, a “symptomatic anti-Parkinsonian therapy” refers to a non-fecal microbiome based therapy. Examples of such therapy include any of bromocriptine, benztropine, levodopa, ropinirole, pramipexole, rotigotine, cabergoline, entacapone, tolcapone, amantidine and selegiline.

As used herein, “reducing the rate of progression of PD” means reducing the deterioration experienced by a PD patient, e.g. as quantified by UPDRS score, as compared to the deterioration experienced by a PD patient not receiving a treatment provided here over a period of time.

As used herein, “delaying the need for symptomatic anti-Parkinsonian therapy” means delaying the need for a symptomatic anti-Parkinsonian therapy for a PD patient who receives fecal microbiome based therapy, as compared to a patient not receiving fecal microbiome based therapy.

As used herein, “early signs of PD” refer to one or more of the followings:
a) a resting 4- to 8-Hz pill-rolling tremor of one hand;
b) tremor which is maximal at rest, diminishes during movement, and is absent during sleep;
c) rigidity and slowing of movement (bradycardia), decreased movement (hypokinesia), and difficulty in initiating movement (akinesia);
d) the face becoming masklike, with mouth open and diminished blinking, which may be confused with depression;
e) the posture becoming stooped;
f) difficulty in initiating walking; the gait becoming shuffling with short steps, and the arms being held flexed to the waist so as to not swing with the stride;
g) steps occasionally inadvertently quickening, and the patient occasionally breaking into a run to keep from falling (festination);
h) tendency to fall forward (propulsion) or backward (retropropulsion) when the center of gravity is displaced, resulting from loss of postural reflexes;
i) Speech becoming hypophonic, with a characteristic monotonous, stuttering dysarthria;
j) Hypokinesia and impaired control of distal musculature resulting in micrographia and increased difficulty with daily living activities;
k) infrequent blinking and lack of facial expression;
l) decreased movement;
m) impaired postural reflexes; and/or
n) characteristic gait abnormality.

As used herein, an “early stage PD patient” is a PD patient at Stage I or II of the Parkinson's Disease as defined by Hoehn and Yahr, and who does not require symptomatic anti-Parkinsonian therapy. Preferably such PD patient does not require symptomatic treatment for at least the next 9 months. An early stage PD patient may be identified as such by performing relevant testing.

As used herein, “eukaryotic” refers to belonging to a cell that contains a nucleus and membrane-bound organelles.

As used herein, “bacteria,” “bacterium,” and “archaea” refer to single-celled prokaryotes that lack membrane bound nuclei and lack organelles.

As used herein, “colony forming units” (cfu) refers to an estimate of the number of viable microorganism cells in a given sample.

As used herein, “viable” means possessing the ability to multiply.

As used herein, “fecal bacteria” refers to bacteria that can be found in fecal matter.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered, for example, weekly, daily, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

PD is a central nervous system degenerative disorder. It is also known as idiopathic parkinsonism, or idiopathic Parkinson's disease. The cause of the disease remains unknown, but it is manifested by an impairment in neurons in the substantia nigra of the brain, resulting in lack of production of dopamine, a neurotransmitter. The reduction in dopamine levels can cause movement impairments via irregular nerve firing in the brain. Major motor impairments associated with PD include tremor, rigidity, bradykinesia, and postural instability.

Current pharmaceutical treatments for Parkinson's disease focus on dopaminergic agents, which either mimic dopamine, or increase levels of dopamine in the body. The most common therapy is levodopa, which is a metabolic precursor of dopamine. However, long term levodopa therapy is often accompanied by dyskinesias, which are sudden, involuntary movements. Additionally, an “on-off” effect develops with long term levodopa therapy, wherein the patient experiences normal movement during “on” times, and reverts to PD-related motor impairment during “off” times. As levodopa therapy progresses, these side effects increase, and the “off” times last longer. Other dopaminergic agents, anticholinergics and/or amantadine also are used either alone, or in combination with levodopa therapy.

Surgical procedures such as deep brain stimulation have also been employed to alleviate symptoms of Parkinson's disease. Recent studies have shown that certain regions of the brain are hyperactive in PD and may be contributing to PD tremors, and cortical stimulation has been proposed to improve symptoms of PD in a mouse model and in humans

Many chronic diseases and disorders of the GI tract have chronic infection/infestation as their underlying pathological conditions (e.g., PD). In one aspect, the present disclosure includes and relates to the use of a fecal microbiota, one or more microbial species therefrom, an active fragment or component therefrom for the treatment and/or prophylaxis of various disease states (e.g., PD) related to the presence of ‘abnormal’ microflora in the GI tract. An active fragment of a bacterium can be any active molecule isolated from such bacteria by any known method for preparing/identifying active fragments of bacteria and proteins secreted from bacteria. Such methods include but are not limited to the following: sonication, osmotic shock, detergent lysis, high pressure, transfer appropriate DNA to other organisms, such as bacteria, plant or animal that is then used as a feed additive as described previously. In one aspect, an active fragment or component of a bacterium is selected from the group consisting of a mycolate or a derivative thereof, a polysaccharide, a lipoglycan, a small peptide, a thiopeptide, a protein, a nucleic acid molecule, a metabolite, a cell wall component, or any combination thereof. In one aspect, an active fragment is a protein or a secretion. In another aspect, an active fragment is a secreted protein.

In one aspect, the present disclosure provides a method for treating PD in a subject in need thereof, where the method comprises administering to the subject a pharmaceutically active dose of a therapeutic composition comprising live non-pathogenic fecal bacteria and/or a sterile fecal filtrate. In another aspect, this disclosure provides use of a composition comprising live non-pathogenic fecal bacteria and/or a sterile fecal filtrate in the manufacture of a medication for the treatment of PD.

In one aspect, a therapeutic composition comprises an isolated or purified population of live non-pathogenic fecal bacteria. In one aspect, a therapeutic composition comprises a non-selected fecal microbiota. In another aspect, a therapeutic composition comprises a non-selected and substantially complete fecal microbiota. In another aspect, a therapeutic composition comprises a full-spectrum fecal microbiota. In one aspect, besides a fecal microbiome-based therapy, a method further comprises administering to a patient concurrently or sequentially a symptomatic anti-Parkinsonian therapy. In another aspect, besides a fecal microbiome-based therapy, a method further comprises administering to a patient concurrently or sequentially bromocriptine, benztropine, levodopa, ropinirole, pramipexole, rotigotine, cabergoline, entacapone, tolcapone, amantadine, selegiline or a combination thereof.

In one aspect, a fecal microbiome-based PD therapy described here is used in combination with, consecutively, concurrently, or sequentially with another treatment selected from the group consisting of levodopa (with or without carbidopa), dopamine agonists (DAs), inhibitors of enzymes that inactivate dopamine (MAO B inhibitors and COMT inhibitors), anticholinergics, amantadine, and estrogen. In a further aspect, a fecal microbiome-based therapy described here is used in combination with one or more drugs selected from the group consisting of levodopa, a monoamine oxidase B (MAO B) inhibitor (e.g., selegiline and rasagiline), dopamine agonists (e.g., bromocriptine, pramipexole, ropinirole, transdermal rotigotine, and apomorphine given by injection), catechol-O-methyl transferase (COMT) inhibitors (e.g., tolcapone and entacapone), an anticholinergic medication (e.g., trihexyphenidyl, benztropine, orphenadrine, procyclidine, and biperiden), amantadine.

In one aspect, the present disclosure provides a method which eliminates or reduces one or more PD symptoms selected from the group consisting of tremor, slowed movement (bradykinesia), rigid muscles, impaired posture and balance, loss of automatic movements, speech changes, and writing changes. In another aspect, the present disclosure provides a method which eliminates, improves, relieves, or reduces one or more early PD symptoms. In another aspect, a treatment method provided here can reduce, relieve, or control one or more non-motor PD symptoms, e.g., depression, sleep disorder, dementia, psychosis and hallucinations.

In a further aspect, a treatment method provided here can reduce, relieve, or control one or more PD primary motor symptoms selected from the group consisting of tremor, bradykinesia, rigidity, and postural instability. In another aspect, a treatment method provided here can reduce, relieve, or control one or more PD secondary motor symptoms selected from the group consisting of freezing of gait, micrographia (a shrinkage in handwriting), mask-like expression (a person's face may appear less expressive than usual), unwanted accelerations, stooped posture (a tendency to lean forward), dystonia, Impaired fine motor dexterity and motor coordination, impaired gross motor coordination, poverty of movement (decreased arm swing), akathisia, speech problems (such as softness of voice or slurred speech caused by lack of muscle control), difficulty swallowing, sexual dysfunction, cramping, and drooling and excess saliva resulting from reduced swallowing movements. In another aspect, a treatment method provided here can reduce, relieve, or control in a PD patient one or more conditions selected from the group consisting of
Loss of sense of smell, constipation
REM behavior disorder (a sleep disorder)
Mood disorders
Orthostatic hypotension (low blood pressure when standing up).
Sleep disturbances
Constipation
Bladder problems
Sexual problems
Excessive saliva
Weight loss or gain
Vision and dental problems
Fatigue and loss of energy
Depression
Fear and anxiety
Skin problems
Cognitive issues, such as memory difficulties, slowed thinking, confusion and in some cases, dementia and
Medication side effects, such as impulsive behaviors.

In one aspect, a PD treatment described here comprises no, or is substantially free of, one or more side effects selected from the group consisting of nausea, sleepiness, dizziness, headache, vomiting, dry mouth, blurred vision, constipation, difficulty emptying the bladder, impaired sweating, and rapid heart rate, low blood pressure after standing up, confusion, hallucinations, delusions, agitation, swelling in the lower legs and feet, swelling of the ankles, dyskinesia, orange discoloration of the urine, and psychosis.

In one aspect, a patient treated here is pretreated with 7-10 days of oral broad-spectrum antimicrobial therapy. In one aspect, a single-agent antibiotic regimen is used. In another aspect, a multiple-agent antibiotic regimen is used. Exemplary antibiotic regimens include the following: (a) ciprofloxacin and metronidazole; (b) trimethoprim-sulfamethoxazole and metronidazole; (c) moxifloxacin, (d) amoxicillin/clavulanic acid. Further exemplary antibiotics include piperacillin/tazobactam, ampicillin/sulbactam, ticarcillin/clavulanic acid, imipenem, meropenem, tigecycline (when severe penicillin allergy is a concern). In another aspect, a multiple-drug regimen may consist of metronidazole and a third-generation cephalosporin or a fluoroquinolone, such as the following: Ceftriaxone, Cefotaxime, Ciprofloxacin, and Levofloxacin.

In one aspect, the present disclosure provides a method for treating PD in a subject in need thereof, where the method comprises administering to the subject a pharmaceutically active dose of a therapeutic composition comprising live non-pathogenic bacteria. In one aspect, the present disclosure provides a method for treating PD in a subject in need thereof, where the method comprises administering daily or weekly to the subject a pharmaceutically active dose of a therapeutic composition comprising live non-pathogenic fecal bacteria. In one aspect, a therapeutic composition is administered to an PD patient in need thereof at least once daily or at least once weekly for at least two consecutive days or weeks. In one aspect, a therapeutic composition is administered at least once daily or at least once weekly for at least 3, 4, 5, 6, 7, 8, 9, 10, 11, 12, 13, 14, or 15 consecutive days or weeks. In another aspect, a therapeutic composition is administered at least once daily or at least once weekly for at least 1, 2, 3, 4, 5, 6, 7, 8, 9, 10, 11, or 12 consecutive weeks. In one aspect, a therapeutic composition is administered at least once daily or at least once weekly for at most 4, 5, 6, 7, 8, 9, 10, 11, 12, 13, 14, 15, 16, 17, 18, 19, or 20 consecutive days or weeks. In another aspect, a therapeutic composition is administered at least once daily or at least once weekly for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an PD patient in need thereof at least twice daily or at least twice weekly for at least two consecutive days or weeks. In one aspect, a therapeutic composition is administered at least twice daily or at least twice weekly for at least 3, 4, 5, 6, 7, 8, 9, 10, 11, 12, 13, 14, or 15 consecutive days or weeks. In another aspect, a therapeutic composition is administered at least twice daily or at least twice weekly for at least 1, 2, 3, 4, 5, 6, 7, 8, 9, 10, 11, or 12 consecutive weeks. In one aspect, a therapeutic composition is administered at least twice daily or at least twice weekly for at most 4, 5, 6, 7, 8, 9, 10, 11, 12, 13, 14, 15, 16, 17, 18, 19, or 20 consecutive days or weeks. In another aspect, a therapeutic composition is administered at least twice daily or at least twice weekly for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an PD patient in need thereof at least three times daily or at least three times weekly for at least two consecutive days or weeks. In one aspect, a therapeutic composition is administered at least three times daily or at least three times weekly for at least 3, 4, 5, 6, 7, 8, 9, 10, 11, 12, 13, 14, or 15 consecutive days or weeks. In another aspect, a therapeutic composition is administered at least three times daily or at least three times weekly for at least 1, 2, 3, 4, 5, 6, 7, 8, 9, 10, 11, or 12 consecutive weeks. In one aspect, a therapeutic composition is administered at least three times daily or at least three times weekly for at most 4, 5, 6, 7, 8, 9, 10, 11, 12, 13, 14, 15, 16, 17, 18, 19, or 20 consecutive days or weeks. In another aspect, a therapeutic composition is administered at least three times daily or at least three times weekly for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating PD in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily or at least once or twice weekly for at least three consecutive days or weeks. In another aspect, a dose is administered at least once, twice, or three times daily or at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating PD in a subject in need thereof, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, a subject being treated is a subject already with PD. Administration of a disclosed therapeutic composition to clinically, asymptomatic human subject who is genetically predisposed or prone to PD is also useful in preventing the onset of clinical symptoms of PD. A human subject genetically predisposed or prone to PD can be a human subject having a close family member or relative exhibiting or having suffered PD. In another aspect, a subject being treated is a subject in which PD is to be prevented. In another aspect, a subject being treated is predisposed or susceptible to PD. In another aspect, a subject being treated is a subject diagnosed as having PD. In one aspect, a subject being treated is a patient in need of PD treatment. In another aspect, a patient being treated is immunocompromised. In another aspect, a patient being treated has both PD and a gastrointestinal disorder (e.g., chronic constipation, IBD, IBS, or a C. diff infection).

In one aspect, a subject being treated has a stage I, stage II, stage III, stage IV, or stage V PD. In one aspect, a method described here is capable of improving a PD patient's condition so that the patient's PD staging is reclassified by at least one or two stages (e.g., from stage III to stage II or from stage III to stage I). In another aspect, a method described here is capable of preventing a PD patient's condition from worsening so that the patient's PD staging remains the same or deteriorates more slowly. In another aspect, a method described here is capable of reducing the rate of progression of PD in a subject. In a further aspect, a method described here is capable of delaying the need for symptomatic anti-Parkinsonian therapy in a subject. In one aspect, a patient is subject to a treatment described here after being diagnosed with one or more early signs of Parkinson's disease.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a method comprises administering a therapeutic composition orally, by enema, or via rectal suppository. In one aspect, a therapeutic composition administered herein is formulated as an enteric coated (and/or acid-resistant) capsule or microcapsule, or formulated as part of or administered together with a food, a food additive, a dairy-based product, a soy-based product or a derivative thereof, a jelly, flavored liquid, ice block, ice-cream, or a yogurt. In another aspect, a therapeutic composition administered herein is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is lyophilized, pulverized and powdered. It may then be infused, dissolved such as in saline, as an enema. Alternatively the powder may be encapsulated as enteric-coated and/or acid-resistant capsules for oral administration. These capsules may take the form of enteric-coated and/or acid-resistant microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, spray-dried, foam-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In a further aspect, a method further comprises pretreating a subject with an anti-inflammatory drug prior to administration of a therapeutic bacterial or microbiota composition.

In one aspect, a method achieves a remission, cure, response, or resolution rate of PD of at least about 10%, 15%, 20%, 25%, 30%, 35%, 40%, 45%, 50%, 55%, 60%, 65%, 70%, 75%, 80%, 85%, 90%, 95%, 97%, or 99%. In one aspect, a treatment method achieves a reduction of UPDRS score after 4, 8, or 12 weeks of treatment by more than 2, 3, 4, 5, 6, 7, 8, 9, 10, or 11. In another aspect, a treatment method achieves a reduction of UPDRS score after 4, 8, or 12 weeks of treatment by more than 2, 3, 4, 5, 6, 7, 8, 9, 10, or 11 in at least 10%, 20%, 30%, 50%, 60%, 70%, 80%, or 90% patients in a patient population. In one aspect, a treatment method achieves at least 10%, 20%, 30%, 50%, 60%, 70%, 80%, or 90% reduction of UPDRS score after 4, 8, or 12 weeks of treatment compared to baseline (e.g., immediately prior to treatment). In one aspect, a treatment method achieves at least 10%, 20%, 30%, 50%, 60%, 70%, 80%, or 90% reduction of UPDRS score in at least 10%, 20%, 30%, 50%, 60%, 70%, 80%, or 90% patients after 4, 8, or 12 weeks of treatment compared to baseline (e.g., immediately prior to treatment). Though UPDRS is used as an example for characterizing and quantifying PD severity, similar or comparable systems (e.g., MDS-UPDRS) can be used as well. Every instance in this application where UPDRS is mentioned, another similar scale or system can be used in lieu of UPDRS. Such alternative systems include, e.g., UPDRS disability score (UDS) (Martinez-Martin et al., Neurologia. 2000 November; 15(9):382-7). In another aspect, an UPDRS score reduction mentioned herein is a reduction of the total UPDRS score. In another aspect, an UPDRS score reduction mentioned herein refers to a reduction in any one or two of the subscores for 1) Mentation, Behavior, and Mood, 2) Activities of Daily Living (ADL) and 3) Motor sections.

In one aspect, a treatment method described here is capable of achieving a rapid onset of PD symptom improvement. In another aspect, a patient's PD symptom improves after one week of five or fewer fecal microbiome infusions via colonoscopic delivery or enema. In another aspect, after one treatment course described herein, a PD patient experience symptom improvement without 1, 2, 3, 4, 5, 6, 7, 8, 9, 10, 11, 12, 13, 14, or 15 weeks of initiating the treatment.

In another aspect, a PD patient is subject to a fecal microbe-based therapy which includes an induction phase of intensive dosing of fecal microbes followed by a maintenance phase of less intensive dosing. In one aspect, an induction phase comprises fresh fecal microbiome infusions via colonoscopic delivery or enema. In one aspect, an induction phase comprises 5 to 10 fresh fecal microbiome infusions via colonoscopic delivery or enema across 1 to 2 weeks. In another phase, an induction phase comprises ingesting acid-resistant/delayed release capsules containing lyophilized donor-derived, non-selected fecal microbiota. In one aspect, a maintenance phase comprises ingesting capsules containing lyophilized donor-derived, non-selected fecal microbiota. In a further aspect, a maintenance phase comprises daily intake of 6 or more, 4 or more, or 2 or more capsules where each capsule comprises about 10¹⁰ to 10¹³ total cell count. In a further aspect, a maintenance phase comprises intake of 6 or more, 4 or more, or 2 or more capsules every other day where each capsule comprises about 10¹⁰ to 10¹³ total cell count.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², or 10¹³, 10¹⁴, or 10¹⁵ cfu. In another aspect, a pharmaceutically active therapeutic effective dose comprises at most about 10⁵, 10⁶, 10⁷, 10⁸, 10⁹, 10¹⁰, 10¹¹, 10¹², 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cells or spores. In another aspect, a pharmaceutically active or therapeutic effective dose comprises at most about 10⁵, 10⁶, 10⁷, 10⁸, 10⁹, 10¹⁰, 10¹¹, 10¹², 10¹³, 10¹⁴, or 10¹⁵ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter. In an aspect, a pharmaceutically active or therapeutic effective dose comprises between 10¹⁰ and 10¹² cells. In another aspect, a pharmaceutically active or therapeutic effective dose comprises between 10¹⁰ and 10¹² cells per capsule.

In one aspect, a therapeutic composition administered herein comprises fecal bacteria. In one aspect, a therapeutic composition administered herein comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a therapeutic composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In one aspect, a therapeutic composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a therapeutic composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, Desuifomonas pigra, Lactobacillus G, Succinivibrio A, or a combination thereof.

In one aspect, a therapeutic composition administered herein comprises a fecal microbiota. In another aspect, the preparation of a fecal microbiota used herein involves a treatment selected from the group consisting of ethanol treatment, detergent treatment, heat treatment, irradiation, and sonication. In another aspect, the preparation of a fecal microbiota used herein involves no treatment selected from the group consisting of ethanol treatment, detergent treatment, heat treatment, irradiation, and sonication. In one aspect, the preparation of a fecal microbiota used herein involves a separation step selected from the group consisting of density gradients, filtration (e.g., sieves, nylon mesh), and chromatography. In another aspect, the preparation of a fecal microbiota used herein involves no separation step selected from the group consisting of density gradients, filtration (e.g., sieves, nylon mesh), and chromatography. In another aspect, a fecal microbiota used herein comprises a donor's entire fecal microbiota. In another aspect, a therapeutic composition administered herein comprises a fecal microbiota substantially free of eukaryotic cells from the fecal microbiota's donor.

In another aspect, a therapeutic composition administered herein comprises a fecal microbiota further supplemented, spiked, or enhanced with a fecal microorganism. In one aspect, a fecal microbiota is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a therapeutic composition administered herein comprises a fecal microbiota further supplemented, spiked, or enhanced with a species of Veillonellaceae, Firmicutes, Gammaproteobacteria, Bacteroidetes, or a combination thereof. In another aspect, a therapeutic composition administered herein comprises a fecal microbiota further supplemented with fecal bacterial spores. In one aspect, fecal bacterial spores are Clostridium spores, Bacillus spores, or both.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided or administered herein comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In an aspect, a therapeutic composition provided or administered herein comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a therapeutic composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.33 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, 0.002 mm, 0.1 mm, or 0.2 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the colon of a normal healthy human. In an aspect, a therapeutic composition provided or administered herein comprises an extract of human feces where the composition is substantially odorless. In an aspect, a therapeutic composition provided or administered herein comprises fecal material or a fecal floral preparation in a lyophilized, crude, semi-purified or purified formulation.

In an aspect, a fecal microbiota in a therapeutic composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a fecal microbiota in a therapeutic composition comprises a donor's substantially entire or non-selected fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, execreted or otherwise liquid components or a microbiota, e.g., biologically active molecules (BAMs), which can be antibiotics or anti-inflammatories, are preserved, retained or reconstituted in a flora extract.

In one aspect, an exemplary therapeutic composition comprises starting material from a donor from a defined donor pool, where this donor contributes a stool that is centrifuged, then filtered with very high-level filtration using e.g., either metal sieving or Millipore filters, or equivalent, to ultimately permit only cells of bacterial origin to remain, e.g., often less than about 5 micrometres diameter. After the initial centrifugation, the solid material is separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation is now taken and filtered progressively in a filtering, e.g., a Millipore filtering or equivalent systems, to end up with liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a therapeutic composition used here comprises a reconstituted fecal flora consisting essentially of a combination of a purified fecal microbiota and a non-cellular fecal filtrate. In another aspect, a therapeutic composition used here comprises a purified fecal microbiota supplemented with one or more non-cellular non-particulate fecal components. In one aspect, a therapeutic composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based therapeutic composition is used concurrently with a fecal non-cellular filtrate-based therapeutic composition. In another aspect, a patient is treated with a first fecal non-cellular filtrate-based therapeutic composition before being given a second fecal bacteria-based therapeutic composition, or vice versa. In a further aspect, a treatment method comprises three steps: first, antibiotic pre-treatment to non-selectively remove infectious pathogen(s); second, a fecal non-cellular filtrate-based treatment step to further suppress selected infectious pathogen(s); and third, giving the patient a fecal bacteria-based therapeutic composition to re-establish a functional intestinal microbiome.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of PD. In another aspect, a treatment method also effects a change of flora. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu or total cell count). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a therapeutic composition comprises at least about 10⁵, 10⁶, 10⁷, 10⁸, 10⁹, 10¹⁰, 10¹¹, 10¹², or 10¹³ cfu or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cfu or total cell count.

In another aspect, a therapeutic composition comprises at least about 10⁵, 10⁶, 10⁷, 10⁸, 10⁹, 10¹⁰, 10¹¹, 10¹², or 10¹³ cells or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cells or total cell count.

In one aspect, a therapeutic composition is formulated as an oral capsule, microcapsule, tablet, or pill. In another aspect, a capsule, microcapsule, tablet, or pill is adapted for enteric delivery. In a further aspect, a capsule, microcapsule, tablet, or pill is an enteric capsule, microcapsule, tablet, or pill. In another aspect, a capsule, microcapsule, tablet, or pill comprises an enteric coating, is acid resistant, or both.

### EXAMPLES

### Example 1. Preparation of Fecal Microbiota

Fecal microbiota is prepared essentially according to protocols published in US2014/0147417 or WO2014/152484. Summarized below is an exemplary protocol.

Potential fecal microbiota donors are screened according to a list of criteria used to exclude unsuitable donors. Potential fecal microbiota donors are excluded if they have received antibiotics, laxatives, diet pills, immunomodulators or chemotherapy in the preceding three months. Potential fecal microbiota donors are excluded if they have a history of all known infectious diseases, morbid obesity, diabetes, irritable bowel syndrome, inflammatory bowel disease, chronic diarrhea, constipation, colorectal polyps or cancer, a compromised immune system, metabolic syndromes, chronic fatigue syndrome, major GI surgery, or other diseases or conditions potentially associated with specific changes in fecal microbiota. Potential fecal microbiota donors are excluded if they exhibit positive laboratory tests for C-reactive protein, erythrocyte sedimentation rate, hepatitis A, hepatitis B, hepatitis C, human immunodeficiency virus, human T-lymphotropic virus or syphilis. Potential fecal microbiota donors are excluded if they exhibit a positive test for stool ova, parasites or viruses. Potential fecal microbiota donors are excluded if they engage in high-risk sexual behaviors, have been incarcerated, or received any tattoos or body piercings in areas that have had disease epidemics within the past three months.

Donor fecal material (fresh feces) is collected in a sterilized container, and then it is transferred to a blender. Approximately 500-1000 mL 0.9% saline solution is added to the blender and thoroughly mixed with the fecal sample. The resulting suspension is filtered at least 4 times through strainers prior to collecting a final suspension. The final suspension is centrifuged in 50 mL tubes at 1200×g for 3 minutes. The supernatant is discarded and the pellet is gently resuspended in approximately 50 mL of sterile 0.9% saline solution. The centrifugation and resuspension steps are repeated 2 to 4 additional times. Upon the final centrifugation, the supernatant is discarded. If the fecal microbiota is to be used immediately, the resultant pellet is resuspended in 1.5-volumes of 0.9% saline solution by gently mixing. If the fecal microbiota is to be stored, the resultant pellet is resuspended in 10% sterile glycerol and stored at −80 degrees Centigrade. If fecal microbiota are frozen, they are warmed to room temperature prior to administration to a patient. If fecal microbiota is lyophilized, the lyoprotectant formulation and lyophilization procedure follow essentially those described in US 2016/0331791, published Nov. 17, 2016.

### Example 2: Oral Capsule Treatment Protocol (Short Regimen) for PD Patients with Bowel Dysfunction

Patients are divided into four groups (Groups 1 to 4). Group 1 patients are administered a pre-treatment of antibiotics (e.g., Vancomycin, Metronidazole, and Colchicine) until the patient shows signs of improved bowel movements. Group 2 receives no antibiotics. Both Groups 1 and 2 receive a pre-colonoscopy bowel prep followed by capsule fecal microbiome therapy. Groups 3 and 4 receive no bowel prep while Group 3, not group 4, also receive an antibiotic pretreatment. Capsules are administered for 2 weeks as follows: two capsules (each containing 10⁹ to 10¹¹ bacterial organisms), given twice-a-day for 4 days, two capsules twice-a-day every other day for 10 days. High dose capsules (total cell count of about 10¹²) are used in loading doses (also called treatment doses) for the initial 4 days. Lower dose capsules (total cell count of about 10⁹) are used in maintenance doses for the subsequent 10 days. In patients receiving antibiotic pretreatment, capsules are administered one day after ceasing antibiotics. Patient symptoms are observed and clinical examination is performed before, during and post oral capsule treatment. Pre, during and post-treatment DNA metagenomics (2-4 days; 1 week; 6 weeks; 12 weeks) are also carried out.

### Example 3: Oral Capsule Treatment Protocol (Intermediate Regimen) for PD Patients with Bowel Dysfunction

Patients are divided into four groups (Groups 1 to 4). Group 1 patients are administered a pre-treatment of antibiotics (e.g., Vancomycin, Metronidazole, and Colchicine if constipation is an issue) until the patient shows signs of improved bowel movements. Group 2 receives no antibiotics. Both Groups 1 and 2 receive a pre-colonoscopy bowel prep followed by capsule fecal microbiome therapy. Groups 3 and 4 receive no bowel prep while Group 3, not group 4, also receive an antibiotic pretreatment. Capsules are administered for 18 weeks or more, as follows: two capsules twice-a-day for 14 days, two capsules twice-a-day every other day for 14 days, 4 capsules twice-a-week for 14 days, and 4 capsules once-a-week (e.g., each Monday) for 12 weeks. High dose capsules (total cell count of about 10¹²) are used in loading doses (also called treatment doses) for the initial 4 weeks. Lower dose capsules (total cell count of about 10⁹) are used in maintenance doses for the subsequent 14 weeks. In patients receiving antibiotic pretreatment, capsules are administered one day after ceasing antibiotics. Patient symptoms are observed and clinical examination is performed before, during and post oral capsule treatment. Pre, during and post-treatment DNA metagenomics (2-4 days; 1 week; 6 weeks; 12 weeks) are also carried out.

### Example 4: Treatment of Patient with PD

A 59-year old patient with PD is treated with a fecal microbiome-based therapy based on acid resistant/delayed release double encapsulated oral capsules containing lyophilized donor-derived non-selected fecal microbiota. The patient's symptoms include severe bloating, severe urgency, stiffness in left arm, imbalance issues, inability to turn head whilst walking, and inability to focus. The patient experiences one bowel movement per day. The patient is subject to a 8-week treatment schedule which includes 10 fecal microbiome therapy liquid rectal enema infusions during a two week induction period, followed by 4 capsules (total cell count of each capsule: about 10⁹-10¹²) per day during a 6 week maintenance treatment period. At week three post-initial treatment, the patient no longer experiences bloating, has increased energy, slight mobility in left arm, and is able to get out of bed without the use of prescribed PD medication. The patient continues to feel heart burn. The patient experiences 2 bowel movements per day. At week four post-initial treatment, the patient experiences seconds of urgency, is unable to get out of bed without prescribed PD medication, experiences PD symptoms, chalky patterns on palm of hands, and severe headaches. The patient experiences 2-3 bowel movements per day. At week five post-initial treatment, the patient experiences mild urgency, dry heaving, increased use of hand gestures, flatulence and burping, and overall improvement in movement (right arm insignificantly improved and left arm stiffness is still present). Patient's overall view on life has improved. The patient experiences one bowel movement per day. The Patient's condition remains improved at week 7 to a similar extent as in week 5. Patient ceases treatment due to a thyroidectomy at week 8.

### Example 5: Treatment of Patient with PD

A 55-year old patient with PD is treated with a fecal microbiome-based therapy based on acid resistant/delayed release double encapsulated oral capsules containing lyophilized donor-derived non-selected fecal microbiota. The patient's symptoms include mild bloating, mild urgency, mild abdominal discomfort, sensory loss, tightness in throat. Patient experienced prior symptoms of sensory loss, tightness in throat, tremor in left ring finger, frozen left shoulder, prolonged cough, stiffness in left arm, severe constipation, pain prior to defecation, and tremor of left leg. The patient experiences two bowel movements per day. The patient is started on a 7 week treatment plan which includes 5 fecal microbiome colonscopic infusions during an induction period of 1 week and 4 capsules/day for 6 weeks during a maintenance period. At week 5 post initial fecal microbiome therapy, the patient experiences no bloating, abdominal discomfort, or urgency. Further some PD symptom improvements are also observed. For example, tremor in left leg and arm decreases. However, a decrease in balance is also observed. At week 6, no major improvements in PD symptoms are observed.

### Example 6: Treatment of Patient with PD

A 60-year old patient with PD is treated with a fecal microbiome-based therapy. The patient's symptoms include speech, hand tremor, difficulty walking, and micrographic writing. The patient is prescribed with a 8 week treatment plan which includes 10 fresh fecal microbiome infusions during an induction period of 2 weeks and 4 capsules/day for 6 weeks during a maintenance period.

As various modifications could be made in the constructions and methods herein described and illustrated without departing from the scope of the disclosure, it is intended that all matter contained in the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entirety.

1. A method for treating Parkinson's disease (PD) in a human subject in need thereof, said method comprising orally administering to said human subject a pharmaceutically active dose of a therapeutic composition comprising live non-pathogenic fecal bacteria.

2.-90. (canceled)

91. The method of claim 1, wherein said subject has a PD at a clinical stage selected from the group consisting of Stages I to V.

92. The method of claim 1, wherein said therapeutic composition comprises a non-selected fecal microbiota.

93. The method of claim 92, wherein said fecal microbiota comprises a donor's entire fecal microbiota.

94. The method of claim 92, wherein said fecal microbiota is from synthetic fecal material.

95. The method of claim 1, wherein said method reduces the rate of progression of PD in said human subject.

96. The method of claim 1, wherein said dose is administered at least once daily for at least 2 weeks.

97. The method of claim 1, wherein said dose is administered at least twice weekly for at least two weeks.

98. The method of claim 97, wherein said dose is administered at least twice weekly for at least 4 weeks.

99. The method of claim 97, wherein said dose is administered at least twice weekly for at least 8 weeks.

100. The method of claim 1, wherein said dose is administered at least three times weekly for at least 4 weeks.

101. The method of claim 1, wherein said therapeutic composition comprises both live non-pathogenic fecal bacteria and a non-cellular fecal filtrate.

102. The method of claim 1, wherein said method comprises a first dosing schedule followed by a second dosing schedule, wherein said second dosing schedule comprises a maintenance dose lower or equal to the dose of said first dosing schedule.

103. The method of claim 1, wherein said therapeutic composition is formulated as a delayed or gradual enteric release form.

104. The method of claim 1, wherein said therapeutic composition is formulated as an enteric coated capsule or an acid-resistant capsule.

105. The method of claim 1, wherein said pharmaceutically active dose is from 10⁸ to 10¹⁴ cfu or total number of cells.

106. The method of claim 1, wherein said therapeutic composition is in a liquid, frozen, freeze-dried, spray-dried, foam-dried, or powder form.

107. The method of claim 1, wherein said therapeutic composition comprises a cryoprotectant selected from the group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, and a combination thereof.

108. The method of claim 1, wherein said subject is pretreated with an antibiotic prior to administration of said composition.

109. The method of claim 1, wherein said method eliminates or reduces one or more PD symptoms selected from the group consisting of tremor, slowed movement (bradykinesia), rigid muscles, impaired posture and balance, loss of automatic movements, speech changes, and writing changes.
